(19) United States
(12) Patent Application Publication
(10) Pub. No.: US 2010/0190160 A1
(43) Pub. Date: Jul. 29, 2010

(54) INDICATOR CELL LINES AND METHODS FOR MAKING SAME

(75) Inventors: Peter J. Hurlin, Portland, OR (US); Sandra Fernandez, Portland, OR (US); David Russell, Seattle, WA (US)

Correspondence Address: FISH & RICHARDSON PC, P.O. BOX 1022, MINNEAPOLIS, MN, 55440-1022, US

(73) Assignee: SHRINERS HOSPITALS FOR CHILDREN, Tampa, FL (US)

(21) Appl. No.: 12/524,264

(22) PCT Filed: Apr. 10, 2007
(86) PCT No.: PCT/US2007/066346

§ 371 Date: Nov. 6, 2009

Related U.S. Application Data
(60) Provisional application No. 60/897,166, filed on Jan. 24, 2007.

(51) Int. Cl. C12Q 1/68; C12N 5/10

(52) U.S. Cl. 435/6; 435/366

(57) ABSTRACT

Methods of making an indicator cell are described. The methods include, e.g., contacting a vertebrate cell comprising a functional endogenous target gene under control of an endogenous inducible promoter with a parvoviral vector comprising a construct comprising a targeting DNA sequence linked to a DNA encoding a reporter gene, wherein the construct enters the cell and undergoes homologous recombination with the target gene, thereby operably linking the reporter gene and the target gene; inducing expression of the target gene thereby causing expression of the reporter gene; and selecting the cell based on expression of the reporter gene.

### RELATED APPLICATIONS

This application claims priority to U.S. Patent Application Ser. No. 60/897,166, filed on Jan. 24, 2007, the entire contents of which are hereby incorporated by reference.

### STATEMENT REGARDING FEDERALLY SPONSORED RESEARCH OR DEVELOPMENT

The U.S. Government may have certain rights in this invention pursuant to Grant No. DK55759 awarded by National Institutes of Health.

### TECHNICAL FIELD

This invention relates to generating indicator cell lines using homologous recombination methods and parvoviral vectors.

### BACKGROUND

Gene regulation is a complex process that involves binding and recruitment of transcription factors to a wide variety of regulatory elements, including distal enhancers and proximal promoter sequences. Understanding mechanisms of gene regulation has broad therapeutic implications for human disease. Engineered indicator cell lines are useful, e.g., for monitoring desired gene activity and screening for compounds capable of modulating such activity.

Studies of gene regulation often employ artificial plasmid reporter systems that fuse regulatory regions to cDNAs that encode reporter genes, such as luciferase or chloramphenicol acetyl transferase. Although it is well-established that gene regulatory elements can function at long distance (>50 kb) and may exist 5′, 3′ or be intergenic, most plasmid-based reporter systems require that only a relatively small DNA fragment(s) be used. Furthermore, analysis of regulatory regions or presumptive regions can be compromised in plasmid reporter systems, whether in vivo or in vitro, by the artificial regulatory environment cultivated in this approach. While these limitations can be overcome by analysis of reporter genes that have been introduced into endogenous loci by plasmid-based homologous recombination (Sedivy et al., Trends Genet. 1999 March; 15(3):88-90), efficient generation of reporter cells by this method can be hampered by low efficiency of homologous recombination. This application features a method that facilitates efficient generation of indicator (or reporter) cell lines, which more accurately reflect endogenous gene regulation.

Some studies have shown high efficiency gene targeting with the use of recombinant Adeno-Associated Virus (rAAV) mediated homologous recombination in human somatic cells (Hirata et al., J. Virol. 2000 May; 74(10):4612-20, Hirata et al., Nat. Biotechnol. 2002 July; 20(7):735-8, Porteus et al., Mol Cell Biol. 2003 May; 23(10):3558-65). AAV is a single-stranded DNA parvovirus with a 4.7 kb genome flanked by two inverted terminal repeats (ITR) (Vasileva et al., Nat Rev Microbiol. 2005 November; 3(11):837-47). The efficiency of rAAV targeting surpasses traditional plasmid-based methods by several fold (Bunz F., Curr Opin Oncol. 2002 January; 14(1):73-8, Topaloglu et al., Nucleic Acids Res. 2005 Oct. 7; 33(18):e158). A number of rAAV-mediated targeting strategies have been employed (Topaloglu et al., Nucleic Acids Res. 2005 Oct. 7; 33(18):e158, Hirata et al., J. Virol. 2000 May; 74(10):4612-20, Hirata et al., Nat. Biotechnol. 2002 July; 20(7):735-8, Kohli et al., Nucleic Acids Res. 2004 Jan. 2; 32(1):e3, Vasileva et al., Nucleic Acids Res. 2006 Jul. 5; 34(11):3345-60. Print 2006), and the general features of the rAAV targeting virus include viral ITRs with introduced homologous arms that mediate recombination to specific genomic sites flanking a drug selection cassette. Importantly, the small packaging size of AAV (4.7 kb) restricts the amount of exogenous DNA that can be included in rAAV gene targeting vectors (Vasileva et al., Nat Rev Microbiol. 2005 November; 3(11):837-47). For example, when a typical drug selection cassette, such as the about 1.7 kb PGK-Neo-pA, is used in the targeting vector, there remains space for inclusion of only about 3 kb of targeting sequence, which is usually arranged as homology arms of at least 1 kb. The high efficiency targeting of rAAV occurs despite the relatively small amounts of homologous sequence that can be included. However, the inclusion of homologous arms together with a drug selection cassette can restrict the addition of other sequences, such as reporter genes, larger than about 850 bps into the rAAV targeting vector. While it is possible to decrease the size of homologous arms to increase the size of the inserted sequences (to larger than about 850 bps), such decrease can result in lower targeting rates (Hirata et al. J. Virol. 2000 May; 74:4612-20).

### SUMMARY

This invention is based, inter alia, on the discovery that parvoviral vectors, e.g., recombinant adeno-associated vectors (rAAV), can be used to target, deliver, and operably link various reporter genes, e.g., fluorescent protein genes (e.g., green fluorescent protein, enhanced green fluorescent protein (EGFP), and/or red fluorescent protein), or reporter fusion genes, e.g., EGFP-Luciferase, with endogenous target genes, e.g., c-Myc oncogenes, through homologous recombination. Using the same gene as both a selectable marker and a gene expression reporter overcomes size limitations of parvoviral vectors. Such parvoviral vectors can be designed to carry targeting DNA sequence(s) flanking the reporter gene sequence, allowing homologous recombination at a target sequence. Cells generated using these methods, e.g., primary cell lines (such as fibroblasts, e.g., human fibroblasts), are useful, for example, in monitoring target gene activity and/or expression for the purpose of identifying new drugs.

In one aspect, methods of making an indicator cell are provided. The methods include: (a) contacting a vertebrate cell comprising a functional endogenous target gene under control of an endogenous inducible promoter with a parvoviral vector comprising a construct comprising a targeting DNA sequence linked to a DNA encoding a reporter gene, wherein the construct enters the cell and undergoes homologous recombination with the target gene, thereby operably linking the reporter gene and the target gene; (b) inducing expression of the target gene thereby causing expression of the reporter gene; and (c) selecting the cell based on expression of the reporter gene.

Embodiments can include one or more of the following features.

Inducing target gene expression can include contacting the cell with a compound, e.g., a small molecule, a peptide, a growth factor, a drug, or an antibody or fragment thereof.

Selecting the cell can include evaluating expression of the reporter gene, e.g., to determine whether the reporter is expressed as required for a particular application. Expression of the reporter gene can be further correlated with the expression of the target gene. Selecting can be performed using methods such as fluorescent-activated cell sorting (FACS), light microscopy, and/or drug selection.

The cell can be any cell that is amenable to infection. For example, the cell can be a stem cell or a somatic cell. The cell can be mammalian, e.g., a human cell. The cell can be from various organs and/or tissues, as further described herein. The cell can be a primary cell, an immortalized cell, a fibroblast, an endothelial cell, an epithelial cell, or a white blood cell.

Any gene can be targeted, for example, the target gene can be a cell cycle gene, a DNA-damage checkpoint gene, a gene that causes cancer when overexpressed, a gene involved in cellular senescence, a gene involved in longevity and metabolism, a gene involved in apoptosis, and/or a gene involved in stem cell formation and function.

The reporter gene can encode a fluorescent protein, green fluorescent protein, red fluorescent protein, enhanced green fluorescent protein, luciferase, or a beta-galactosidase.

The parvoviral vector can be, e.g., an adeno-associated viral vector, e.g., an adeno-associated viral 2 vector.

In another aspect, the disclosure features an indicator cell comprising a functional endogenous target gene and an exogenous reporter gene, wherein both the target gene and the reporter gene are under control of an endogenous inducible promoter of the target gene.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification will control. In addition, the materials, methods, and examples are illustrative only and not intended to be limiting. Other features and advantages of the invention will be apparent from the following detailed description, and from the claims.

### DESCRIPTION OF DRAWINGS

FIG. 1 is a diagram depicting an exemplary scheme of direct selection of targeted EGPF-Luciferase reporter gene insertion into the c-Myc locus using known c-Myc regulatory conditions. As shown, subconfluent primary HFF cells are infected with rAAV targeting vectors when c-Myc is being actively transcribed or replicated in proliferative cells. Background reporter gene expression is silenced through density arrest and serum withdrawal. Reporter gene expression is induced by serum stimulation. Single EGFP-Luciferase positive cells are selected using FACS. Cloned cells are expanded and screened for gene targeting events by PCR, Southern blot, and sequencing.

FIG. 2A depicts a FACS histogram for wildtype HFF cells following cell cycle entry (CCE). FL1-A (EGFP) was plotted against FL2-A (no fluorophore) to center the parent population and allow for selection of any EGFP-Luciferase positive cells. The P2 region of the plot indicates the gating used for selection of any EGFP-Luciferase positive cells.

FIG. 2B depicts a FACS histogram for c-Myc rAAV targeted cells following cell cycle entry (CCE). FL1-A (EGFP) was plotted against FL2-A (no fluorophore) to center the parent population and allow for selection of the dim EGFP-Luciferase positive cells. The P2 region of the plot indicates the gating used for selection of the EGFP-Luciferase positive portion of the parent population.

FIG. 3A is a diagram of the c-Myc rAAV targeting and genotype strategies. The targeting region of the endogenous c-Myc locus is shown. The left homologous arm (LHA), right homologous arm (RHA), and inverted terminal repeats (ITR) portions of the rAAV vectors facilitate insertion of the EGFP-Luciferase fusion gene directly between the first and second codon of the c-Myc gene. K represents KpnI; X represents XbaI; C represents ClaI; and S represents SspI.

FIG. 3B is a photomicrograph displaying the results of a triple primer PCR genotyping of EGFP-Luciferase positive clonal populations performed using primers 1-3 shown in FIG. 3A.

FIG. 3C is a photomicrograph displaying the results of a Southern blot analysis using the c-Myc exon 3 probe of FIG. 3A.

FIG. 3D is a schematic showing sequence data of regions spanning the c-Myc rAAV insertion sites and the internal EGFP-Luciferase insertion regions: c-Myc intron 1 and LHA (SEQ ID NO:1); LHA and EGFP (SEQ ID NO:2); Luciferase and RHA (SEQ ID NO:3); and RHA and c-Myc intron 2 (SEQ ID NO:4). LHA represents left homologous arm; RHA represents right homologous arm.

FIG. 4A depicts a histogram of unstimulated MR1 clonal population. The EGFP-Luciferase expressing cells are located in the P2 gated region.

FIG. 4B depicts a histogram of MR1 clonal population's response to cell cycle entry. MR1 was subjected to cell cycle entry conditions (serum-stimulated) and then evaluated for EGFP-Luc reporter gene expression. The EGFP-Luciferase expressing cells are located in the P2 gated region.

FIG. 4C depicts a histogram of unstimulated MR2 clonal population. The EGFP-Luciferase expressing cells are located in the P2 gated region.

FIG. 4D depicts a histogram of MR2 clonal population's response to cell cycle entry. MR1 was subjected to cell cycle entry conditions (serum-stimulated) and then evaluated for EGFP-Luc reporter gene expression. The EGFP-Luciferase expressing cells are located in the P2 gated region.

FIG. 5A is a photomicrograph of the results of an RT-PCR tracking the MR1 cells through cell cycle entry for their ability to induce c-Myc transcript.

FIG. 5B is a photomicrograph of the results of an RT-PCR tracking the MR1 cells through cell cycle entry for their ability to induce EGFP-Luciferase transcript.

FIG. 5C is a photomicrograph of the results of a Western blot tracking the MR1 cells through cell cycle entry for their ability to induce c-Myc transcript. Max protein expression was used as a loading control.

FIG. 5D is a photomicrograph of the results of a Western blot tracking the MR1 cells through cell cycle entry for their ability to induce EGFP-Luciferase transcript. Max protein expression was used as a loading control.

FIG. 5E is a graph of luciferase activity of the MR1 population over the cell cycle entry. Data shown is the average of triplicate samples +/−SEM.

FIG. 5F is a graph of FACS Calibur data where the EGFP-Luciferase positive percent of the parent population was determined using BD CELLQUEST PRO™ v. 5.2. Time course changes of the percent EGFP-Luciferase expressing populations were compared under the same gates.

### DETAILED DESCRIPTION

The present application describes methods for generating indicator cells, e.g., human cells and/or cell lines, that can serve as reporters of, e.g., transcriptional activity. The methods exploit the ability of parvoviral vectors, e.g., recombinant adeno-associated viruses (rAAV), to mediate insertion of exogenous DNA sequences into specific genomic loci through homologous recombination. To overcome the size limitations of some parvoviral vectors, a gene, e.g., a fluorescent protein gene (for example, EGPF, or a fusion gene, e.g., EGFP-Luciferase gene), can act as both a selectable marker and a gene expression reporter, thereby making it unnecessary to use separate markers and reporters. These methods can be used to produce an indicator cell, e.g., a primary, somatic, and/or stem indicator cell. An indicator cell produced as described herein can be used in high-thoroughput screens for compounds, e.g., cDNA, siRNA, and/or small molecules, that modulate endogenous gene expression.

Parvoviral Constructs

The methods described herein utilize parvoviral, e.g., rAAV, gene targeting vectors. The vectors are constructed to carry a promoter-less reporter gene(s). The vectors can carry two viral inverted terminal repeats (ITR) with homologous arms from appropriate sections of the target gene. The homologous arms can mediate homologous recombination to specific sites of the target gene, thus acting as targeting sequences. When rAAV vector is used, relatively small amounts of homologous sequence from the target gene can be included, as the vector has a high efficiency of targeting. General techniques for construction of rAAV vectors for gene targeting via homologous recombination are known in the art. The constructs described herein can be introduced into known packaging cell lines that will produce targeting vectors. Targeting vectors produced by the packaging lines can be collected and used to infect cells.

Target Genes

A variety of endogenous target genes can be operably linked to a reporter gene(s) using the methods described herein. Any inducible gene can be targeted. The target gene can be functional and endogenously expressed in the cells being manipulated. A target gene can include an endogenous inducible promoter, e.g., operably linked such that it can drive the co-expression of the reporter gene and the target gene. Exemplary target genes include, e.g., cell cycle genes and DNA-damage checkpoint genes, e.g., p53, p19ARF, MDM2, p16INK4B, p21Kip1, ATM, ATR, BRCA1 and 2, Cyclins A, B, D and E; genes that cause cancer when overexpressed, e.g., MDM2, Fos, Jun, c-Myc, N-Myc and L-Myc, Tert, Bcl2; genes involved in cellular senescence (many of the same as listed above); genes involved in longevity and metabolism, e.g., SirT family genes, IGF and IGF receptors; genes involved in apoptosis, e.g., Bcl2, BclXL, Caspases, Fas and Fas ligand; and genes involved in stem cell formation and function, e.g., BMP, SHH, Wnt, Notch, and FGF family ligands and their receptors.

Reporter Genes

A variety of reporter gene(s) can be used in the methods described herein. In some instances, a reporter gene may lack a promoter, allowing it to be driven by the endogenous inducible promoter of the target gene. The reporter gene can be a fusion gene, e.g., a EGFP-Luc gene. Exemplary reporter genes are known in the art and can include, e.g., fluorescent proteins, e.g., GFP, EGFP, red fluorescent protein, luciferase; LacZ (βgal), and others.

Construction of an Indicator Cell

The targeting vectors described herein, e.g., rAAV vectors, can be used to generate indicator cell lines by, e.g., infection methods known to those skilled in the art. The resulting indicator cell lines carry an exogenously provided reporter gene under the control of the target gene promoter. Such cells can be selected, e.g., by stimulating or inducing the expression of the target gene, and sorting, e.g., by fluorescence activated cell sorting (FACS), the cells for expression of the reporter gene. The expression of the reporter gene can be directly correlated with the expression of the target gene.

For example, the targeting vectors produced by the packaging cell lines can be titered to a desired concentration and introduced into the medium of a cell. The cell can be at a particular cell cycle stage, for example at a euchromatic state due to active replication or transcription. Homologous recombination is most efficiently facilitated by, e.g., rAAV, when a cell is in a euchromatic state. Target cells can be kept in a medium with targeting vectors for a period of time and then given fresh medium. To select cells into which the reporter gene has been correctly inserted, the cells can be stimulated with a compound or a condition known to stimulate the target gene. The compound can be, e.g., a small molecule, a peptide, a growth factor, a drug, or an antibody or a fragment thereof. The condition can be, inter alia, increased or decreased temperature, increased or decreased osmolarity, increase in cell age, and/or cell cycle entry or exit, among others. Stimulated cells can then be selected by any known method depending on the type of reporter gene used and the expression. For example, if the reporter gene is a fluorescent marker, the cells can be sorted with a FACS machine.

Alternatively or in addition, insertion of the reporter gene can be analyzed by genotype analysis of the indicator cells selected by, e.g., FACS, as described above. For example, genomic DNA can be isolated and analyzed by, e.g., PCR and Southern blot, to determine the size and location of integrated reporter gene. Genomic DNA can be analyzed to determine whether any random integration of the reported gene has occurred.

Expression of the endogenous target gene can be compared with expression of the reporter gene. Expression of the target gene can be analyzed by, e.g., RT-PCR and/or Western blot analysis. Expression of the reporter gene can be analyzed by e.g., RT-PCR, Western blot analysis and/or fluorescent activity (if the reporter gene encodes a fluorescent molecule). Expression of both genes can be correlated, making the indicator cell useful for screening various compounds that may modulate expression of the target gene.

Indicator Cells

A wide variety of cells can be used to generate indicator cells described herein. For example, the cell can be a stem cell or somatic cell, or primary or immortalized, e.g., derived from a tumor. It can be, e.g., mammalian, e.g., human, murine, simian, equine, bovine, porcine, feline, or canine, or combinations thereof. It can be derived from any organ system, e.g., circulatory, skeletal, immune, respiratory, urinary, reproductive, central nervous, peripheral nervous, skin, oral tissues, gastrointestinal tract, liver, pancreas, endocrine glands, and/or sense organs. It can be derived from any tissue type, e.g., blood, muscle, nervous tissue, connective tissue, and/or epithelial tissue. It can be an endothelial, an epithelial, or a neuronal cell; it can be a fibroblast, or a white blood cell, among many others.

Screening Methods

Indicator cells produced with the methods described herein can be useful in screening assays, e.g., to identify and/or analyze potential pharmacological agents. The cells can be used, e.g., to identify new pharmacological agents from a library of test compounds and/or characterize mechanisms of action and/or side effects of compounds that have known pharmacological activities. For example, an indicator cell can be stimulated with a test compound and the activity of the reporter gene analyzed. The activity of the reporter gene may be correlated with the activity of the target gene. The effect of a test compound on, e.g., the induction of the reporter gene, can provide an indication of its effect on, e.g., the induction of the target gene.

Test compounds can include a variety of molecules, e.g., small molecules, peptides, drugs, siRNA, antisense oligonucleotides (e.g., cDNA), antibodies (or fragments thereof), growth factors, and/or combinations thereof.

Assays or tests involving determination of the effect(s) of a test compound can involve determining or comparing the effect(s) of the absence of the test compound, the presence of a positive and/or negative control compound, and/or the presence of one or more test compounds. Typically, such assays or tests involve determining pharmacological properties of the test compound(s).

Test compounds can be obtained in many different ways, e.g., using any of the numerous approaches in compound library methods known in the art. For example, commercially available compound libraries can be used, as can libraries constructed from commercially available compounds, custom compound libraries, synthetic compound libraries, and natural product libraries, e.g., produced by bacteria, yeast, and/or fungi.

One broad category of libraries and library methods are combinatorial library methods including without limitation: biological libraries; spatially addressable parallel solid phase or solution phase libraries; synthetic library methods requiring deconvolution; the “one-bead one-compound” library method; and synthetic library methods using affinity chromatography selection. (Lam, K. S. (1997) Anticancer Drug Des. 12:145). Such libraries can be peptide and/or peptide analog, oligonucleotide and/or oligonucleotide analog, and/or small molecule libraries.

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds may be presented, for example, in solution (e.g., Houghten (1992) Biotechniques 13:412-421), on beads (Lam (1991) Nature 354:82-84), chips (Fodor (1993) Nature 364:555-556), bacteria (Ladner U.S. Pat. No. 5,223,409), spores (Ladner U.S. Pat. No. '409), plasmids (Cull et al. (1992) Proc Natl Acad Sci USA 89:1865-1869) or on phage (Scott and Smith (1990) Science 249:386-390); (Devlin (1990) Science 249:404-406); (Cwirla et al. (1990) Proc. Natl. Acad. Sci. 87:6378-6382); (Felici (1991) J. Mol. Biol. 222:301-310); (Ladner supra.)).

Additional compounds or agents identified according to screening assays can be further tested and/or developed and/or used therapeutically or prophylactically either alone or in combination.

### EXAMPLES

The following examples demonstrate methods for rapidly producing human gene indicator (or reporter) cells by rAAV-mediated gene insertion, and show characterization of a c-Myc gene reporter cell strain developed using these methods. The methods for reporter insertion described herein are applicable to any gene whose expression can be induced from a basal level, and the example provided below is not intended to limit the invention in any way. For example, a number of genes such as cyclins and cell cycle inhibitor/checkpoint genes have well-defined conditions in which their expression can be induced from a low level. Indicator (or reporter) cells for these genes and in particular genes that are involved in cancer and other diseases offer a possible platform for the identification of proteins and molecules with potential therapeutic value.

As further described below, the techniques featured herein were used to develop primary human fibroblasts, in which a promoter-less EGFP-Luciferase (EGFP-Luc) fusion gene has been introduced into the c-Myc locus in frame with, and immediately downstream of, the ATG translational start site (i.e., EGFP-Luc gene was under the control of the c-Myc oncogene). EGFP was then used for selection of correctly targeted alleles by taking advantage of known regulatory conditions that activate transcription of c-Myc.

A. Construction and Packaging of rAAV Gene Targeting Vectors

As further described below, the rAAV vector was designed to insert the 2690 by promoter-less EGFP-Luc fusion gene into exon 2 of the c-Myc locus in frame with the major c-Myc translational start codon. Insertion of the reporter gene was predicted to produce a chimeric transcript comprised of the c-Myc 5′ untranslated region fused to EGFP-Luc. A polyadenylation signal on the EFGP-Luc cDNA prevents read-through transcription of downstream sequences and therefore its insertion inactivates the targeted c-Myc allele. However, the reporter gene knockin was predicted to result in minimal disruption of the native cis regulatory sequences that govern c-Myc transcription so that the reporter gene should provide an accurate readout of endogenous c-Myc activity.

To facilitate production of rAAV targeting vectors that insert an EGFP-Luciferase reporter gene into target loci of interest, the pEGFP-Luciferase cloning vector (pELCV) was created. A promoter-less EGFP-Luciferase-SV40 pA fusion gene was obtained from pEGFPLuc by standard methods. To preserve a unique XbaI site in the final pELCV multiple cloning region, the pEGFPLuc XbaI site in the 3′ portion of the EGFP-Luciferase fusion gene was destroyed by XbaI digestion, Klenow fill-in, and blunt end ligation. The 2690 by EGFP-Luciferase-pA restriction fragment was then removed from pEGFPLuc at NheI and MluI sites and made blunt by a Klenow fill-in reaction. This fragment was blunt end ligated into the SmaI site of pBluescript II SK+. Addition of rAAV ITRs requires DNA fragments with 5′ and 3′NotI ends. Therefore, a second NotI site was introduced into pELCV between the KpnI and XhoI recognition sequences using a KpnI NotI XhoI oligo linker to complement the existing NotI in the pBluescript II SK+ multiple cloning site.

Construction of the c-Myc EGFP-Luc knockin targeting vector was accomplished by PCR amplification of the c-Myc left and right homologous arms from the human genomic BAC RP11 237 F24 (Invitrogen) using primers containing unique exogenous restriction enzyme sites. The 846 by left homologous arm (LHA) was amplified using an EcoRV forward primer (GGTCAGATATCGGAGGAACTGCGAGGAGC) (SEQ ID NO:5) and a PstI reverse primer (CTCGGTCCTGCAGCATCGTCGCGGGAGGCTGCTG) (SEQ ID NO:6) that ends with the c-Myc ATG. The 807 by right homologous arm (RHA) was amplified using a BamHI forward primer (GGTCAGGATCCCCCCTCAACGTTAGCTTCACC) (SEQ ID NO:7) that starts with the first by after the ATG and a XbaI reverse primer (CTCGGTCTAGAGAAGGGATGGGAGGAAACGC) (SEQ ID NO:8). PCR product was restriction digested and sequentially ligated into pELCV. In-frame fusion of the c-Myc ATG with the start codon of the EGFP-Luc fusion gene was confirmed by sequencing. The AAV ITRs were introduced by ligation of the NotI targeting fragment into the pAAV-hrGFP vector backbone. ITR flanked c-Myc targeting vector integrity was confirmed by AhdI restriction mapping. rAAV vector stocks were prepared by standard methods and cotransfection of cells with lipofectamine (Invitrogen). rAAV particles were collected 3 days post-transfection by scraping cells from the 10 cm dish into 1 mL PBS pH 7.4 (Invitrogen) followed by 4 freeze/thaw cycles between a dry ice/ethanol bath and a 37° C. water bath. Vector stocks were clarified by centrifugation and used fresh or stored at −80° C. The titer of the rAAV stock was ˜1×10⁶ viral particles/mL as verified by RT-PCR (Veldwijk et al, 2002 August; 6(2):272-8).

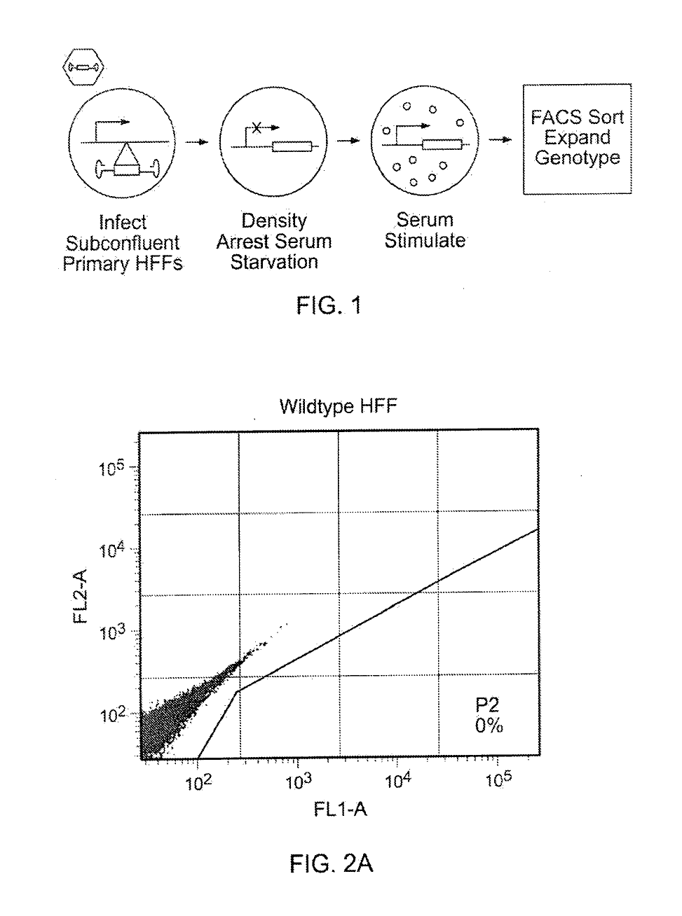

B. Generation of c-Myc gene reporter human fibroblast cell lines Transcription of the c-Myc gene is tightly regulated, and events such as viral transduction, viral integration, chromosomal translocations and gene amplification that deregulate c-Myc transcription can activate its well-characterized oncogenic potential (Grandori et al., Annu Rev Cell Dev Biol. 2000; 16:653-99). In addition, c-Myc mRNA and protein levels are deregulated or elevated in many tumors that show no physical disruption at the gene level. In the latter cases, deregulated c-Myc expression is thought to be due to oncogenic activation of mitogenic signal transduction pathways that regulate c-Myc gene expression (Nesbit et al., Oncogene. 1999 May 13; 18(19):3004-16, Christoph et al., Int J Cancer. 1999 Apr. 20; 84(2):169-73, Erisman et al., Mol Cell Biol. 1985 August; 5(8):1969-76). Indeed, the c-Myc gene has been found to be induced by a wide variety of mitogenic proteins and suppressed by anti-mitogenic proteins (Grandori et al., Annu Rev Cell Dev Biol. 2000; 16:653-99). Although c-Myc transcription is known to be regulated at a variety of levels (Spencer et al., Adv Cancer Res. 1991; 56:1-48), it remains unclear how the various mitogenic and anti-mitogenic signals converge on the c-Myc promoter to control gene expression (Weber et al., Mol Cell Biol. 2005 January; 25(1):147-61). Therefore, to facilitate mechanistic studies of c-Myc gene regulation in human cells, a strategy depicted in FIG. 1 was devised to generate primary human foreskin fibroblasts (HFF) that serve as reporters of c-Myc transcription. This strategy took advantage of the well-documented induction of c-Myc transcription that occurs upon serum stimulation of quiescent cells (Persson et al., Mol Cell Biol. 1985 November; 5(11):2903-12, Rabbitts et al., EMBO J. 1985 August; 4(8):2009-15, Dean et al., J Biol. Chem. 1986 Jul. 15; 261(20):9161-6) to select, by FACS sorting, for rAAV-mediated knockin of an EGFP reporter gene into the c-Myc locus.

Methods

HFF Culture and Cell Cycle Entry Assays

Primary human foreskin fibroblasts (HFFs) were generously provided at passage one from Carla Grandori (Fred Hutchinson Cancer Research Center). HFFs were routinely cultured in high glucose Dulbecco's modified Eagle medium (DMEM) (Invitrogen) supplemented with 10% fetal bovine serum (FBS) (Hyclone), 100 U/mL penicillin, and 100 μg/mL streptomycin (Invitrogen) in incubators at 37° C. and 5% CO₂. For cell cycle entry assays, HFFs were first driven into quiescence by maintaining cells at confluence for 3 days in medium containing 10% FBS followed by 3 days of culture in DMEM containing 0.1% FBS. Quiescent cells were stimulated to enter the cell cycle by the addition of medium containing 20% FBS.

Gene Targeting in Human Fibroblasts

Homologous recombination is most efficiently facilitated by rAAV when target loci are in a euchromatic state due to active replication or transcription (Trobridge et al., Hum Gene Ther. 2005 April; 16(4):522-6, Vasileva et al., Nat Rev Microbiol. 2005 November; 3(11):837-47). Therefore, to bias rAAV integration for homologous targeting instead of random integration events, rAAV was introduced to HFF cultures in log phase proliferation. Primary HFFs at second passage were grown to 40% confluence (˜2×10⁶ cells) in 10 cm plates. HFFs were then infected overnight using 333 μL c-Myc rAAV vector stock at a multiplicity of infection of ˜0.5 in 8 mls fresh medium. Infected cells were given fresh media 24 hours post infection and driven into quiescence by confluence arrest and serum deprivation as described above.

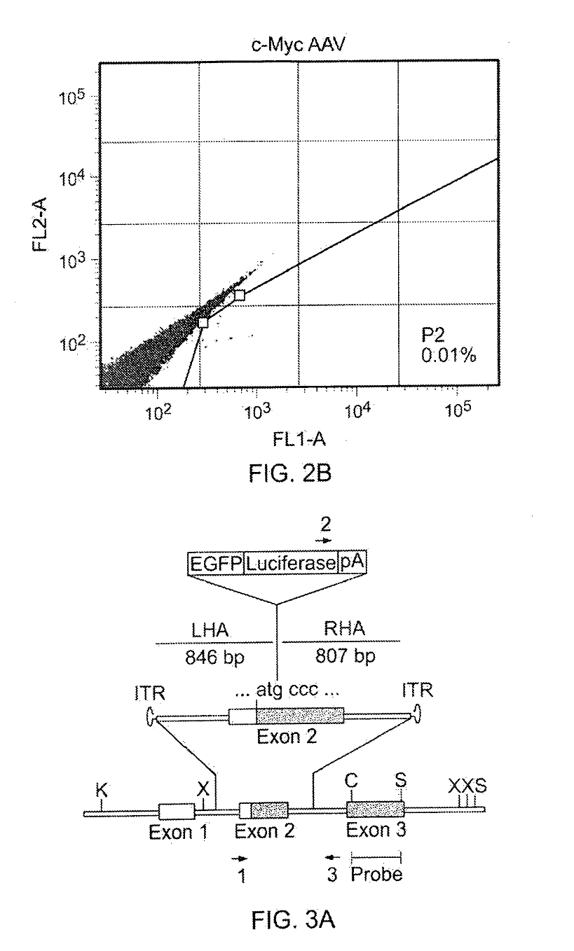

To select infected cells that have the EGFP-Luc gene correctly inserted immediately downstream of the c-Myc translation start site and, therefore, under the control of c-Myc regulation, quiescent cells were stimulated with 20% serum for 6 hours. Stimulated cells were then trypsinized, strained through a 40 μM mesh to generate a single cell suspension at ˜5×10⁶ cells, and then sorted on a FACS Vantage with DiVa (Digital Vantage) upgrade (Becton Dickinson). The FACS Vantage was programmed to deliver single EGFP-Luc positive cells to individual wells of a 96 well tissue culture plate (FIG. 2A). Individual clones were expanded for genotype analysis.

Genotype Analysis

Genomic DNA was isolated from individual EGFP-Luc positive clones. For PCR

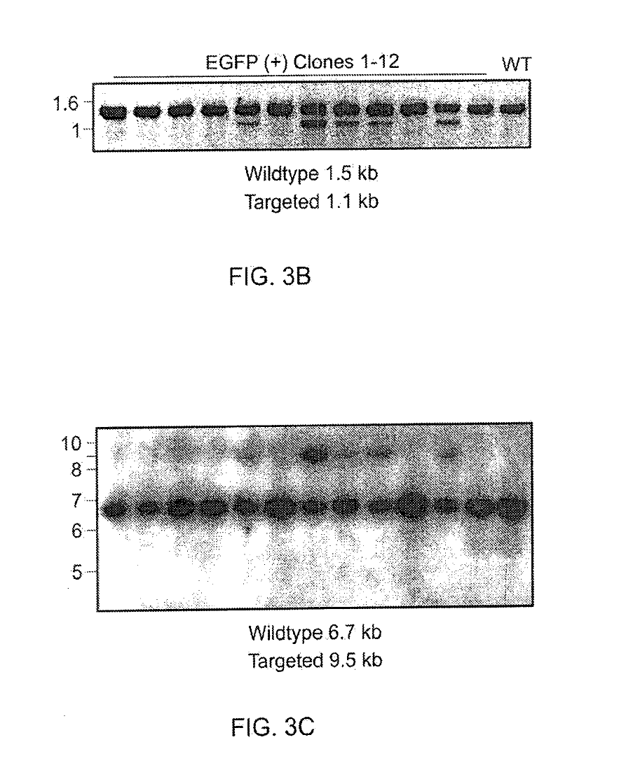

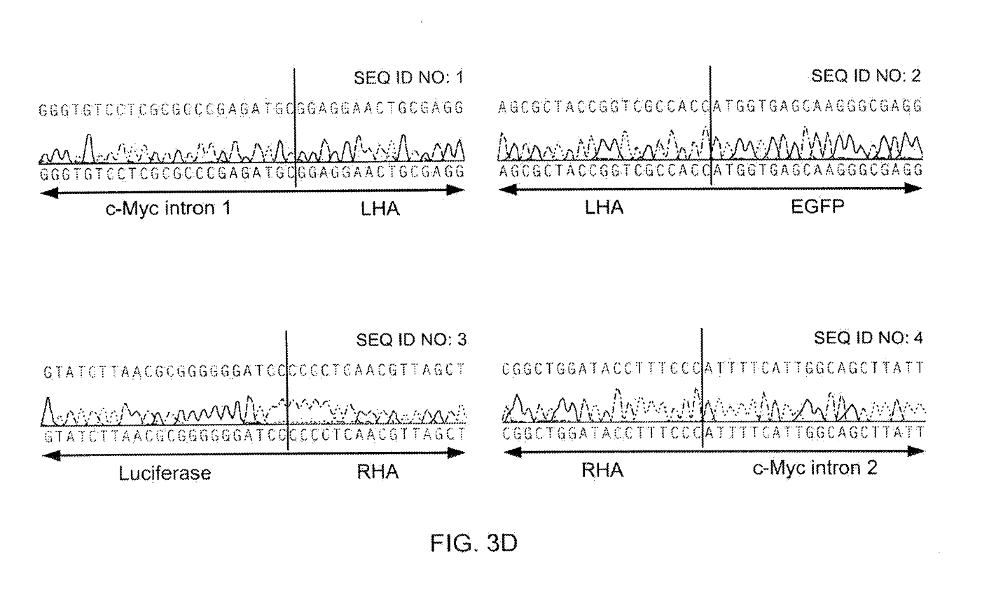

genotyping, triplex PCR was performed using one primer specific for the LHA upstream of the EGFP-Luc insertion site (primer 1), one primer specific to the 3′ end of the EGFPLuc fusion gene (primer 2), and one primer that recognizes sequence 3′ of the RHA that is outside the targeting construct (primer 3) (FIG. 3A). Primers 1 and 3 amplify a 1.5 kb product from the wildtype c-Myc allele. Primers 2 and 3 amplify a 1.1 kb product that indicates the EGFP-Luc fusion gene has been knocked into the c-Myc allele (FIG. 3B). For Southern blot analysis, 20 μg genomic DNA was digested with XbaI overnight, separated on a 0.8% 1×TAE agarose gel, and transferred to Hybond XL nylon membrane
(Amersham). To identify correctly targeted c-Myc alleles membranes were hybridized with an α-³²P dCTP labeled c-Myc exon 3 probe (FIGS. 3A and 3C). To identify random integrations, an EGFP-specific probe was used (data not shown). Targeted clones were finally confirmed by sequencing across the regions where the targeting construct juxtaposes the genomic DNA as well as the EGFP-Luc insert regions within the targeting construct to insure the reporter gene was inserted inframe (FIG. 3D).

Results and Discussion

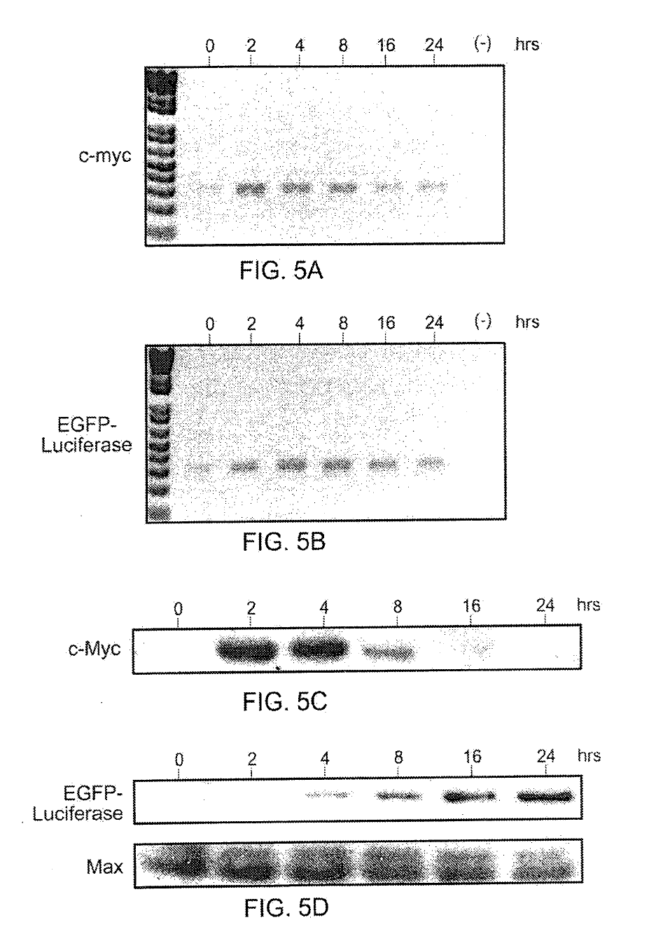

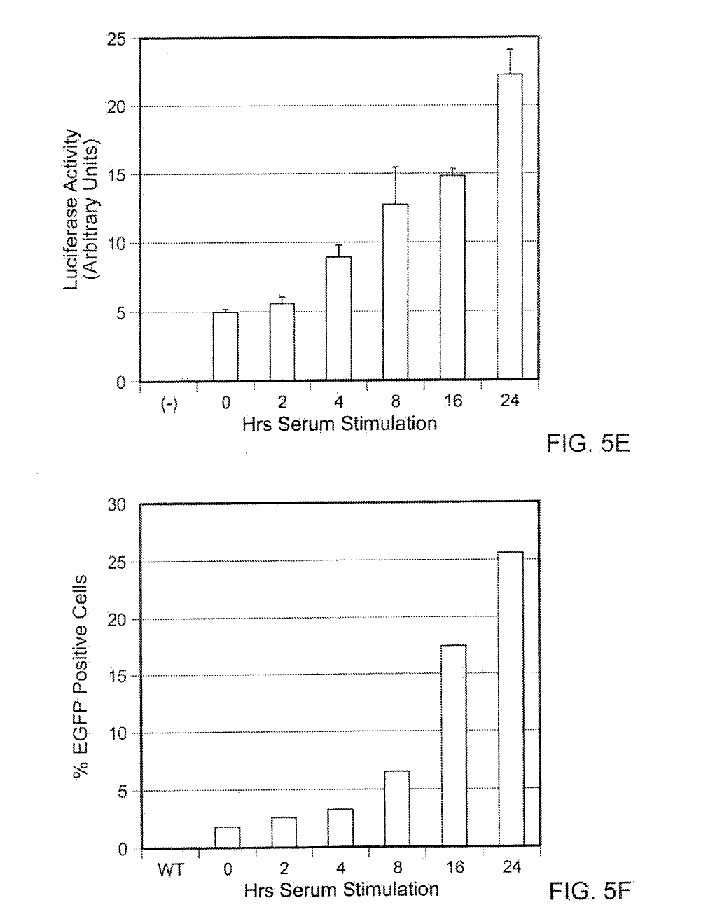

Primary HFFs at passage 2 were infected with the c-Myc rAAV targeting vector for 24 hours. The infected cells were then driven into quiescence by combined confluence arrest and serum deprivation (FIG. 1). These cells were stimulated to reenter the cell cycle by the addition of 20% serum for 6 hours and subjected to FACS sorting for cells that expressed EGFP. Although, c-Myc is maximally induced between 2 and 4 hours following serum stimulation (FIG. 5A-5F), the 6 hour time point was chosen for sorting to ensure that cells had entered the cell cycle and because EFGP-Luc is predicted to have a much longer half life than the short, 20-30 minute half life of c-Myc. Of 5×10⁵ cells sorted, 48 cells (0.01% of the parent population) showed expression of EGFP-Luc (FIG. 2A). EGFP-positive cells were automatically collected and sorted into individual wells of a 96 well dish by the flow cytometry instrument. Of the 48 cells plated, 24 grew out to formed viable colonies. The 24 clonal populations were expanded, their DNA extracted, and PCR and Southern blot genotyping were performed. Five clones (21%) were found to carry a single targeted c-Myc allele (FIGS. 3B and 3C). DNA from these five clones was subjected to sequence analysis to confirm that the EGFP-Luc gene was inserted correctly. All five showed correct in-frame insertion of the reporter beginning after the ATG of the major c-Myc translation start site. Sequence at the insertion junctions for a representative clone is shown in FIG. 3D. These results are consistent with previous results showing that AAV vectors can precisely insert DNA at specific genomic loci (Hirata et al., Nat. Biotechnol. 2002 July; 20(7):735-8).

rAAV-mediated gene targeting has also been shown to lead to random integration events (Hirata et al., Nat. Biotechnol. 2002 July; 20(7):735-8). To determine whether random integration of the EGFP-Luc gene occurred in our selected clones, Southern blots were performed using a probe specific to the EGFP-Luc gene (not shown). This analysis showed three of the clones with random integration events and that Clone 9 had one random integration event (not shown) and one correctly targeted c-Myc allele (FIGS. 3B and 3C). The other 4 clones showed only a single correctly targeted c-Myc allele (FIGS. 3B and 3C) and no random integration events (not shown).

It was found that use of a high titer virus at a high multiplicity significantly improves the percent yield of EGFPLuc positive cells compared to the low titer stocks used in these experiments.

C. Comparison of Endogenous c-Myc Expression to c-Myc Reporter Activity

Methods

RT-PCR

Total RNA was isolated using Trizol reagent (Invitrogen) and cDNA synthesized in a 20 μl reaction from 1 μg total RNA using Superscript III (Invitrogen) and random nonamer primers (Takara). 1 μl cDNA was used as RT-PCR template to detect c-Myc and EGFPLuc mRNA expression. The c-Myc wildtype allele transcripts were detected using a forward primer, GCTCGCCCAAGTCCTGC (SEQ ID NO:9), which anneals in exon 2, and a reverse primer, GCTGATGTGTGGAGACGTGG (SEQ ID NO:10), which anneals in exon 3 (FIG. 5A). EGFPLuc transcripts were detected from a primer pair annealing 3′ in the EGFP-Luc coding region. The EGFP-Luc forward primer is TATGGGCTCACTGAGACTACATCA (SEQ ID NO:11) and the reverse primer is TCAGAGACTTCAGGCGGTCAA (SEQ ID NO:12).

Western Blot

Protein was collected in 50 mM Tris pH 7.4, 1% NP40, 150 mM NaCl, 1 mM EDTA, 1 mM Na3VO4, 1 mM NaF, 1× Complete Protease Inhibitor (Roche). 10 μg of protein was separated by electrophoresis in 4-12% Bis-Tris NuPage gels (Invitrogen) and transferred onto nitrocellulose membrane. Membranes were probed with 1:200 anti-c-Myc (9E10) (Santa Cruz), 1:2000 anti-GFP (JL-8) (Clontech), and 1:500 anti-Max (C-17) (Santa Cruz) antibodies.

Luciferase Assays

Cells were collected from 10 cm dishes into 500 μL Tropix lysis solution supplemented with fresh 0.5 mM DTT. 10 μL of cellular lysate was added to 25 μL Dual Light Buffer A in a 96 well plate. Samples were assayed for Luciferase activity on a Tropix TR717Microplate Luminometer (PE Applied Biosystems). Samples were run in triplicate and the average data are reported +/−SEM.

Results and Discussion

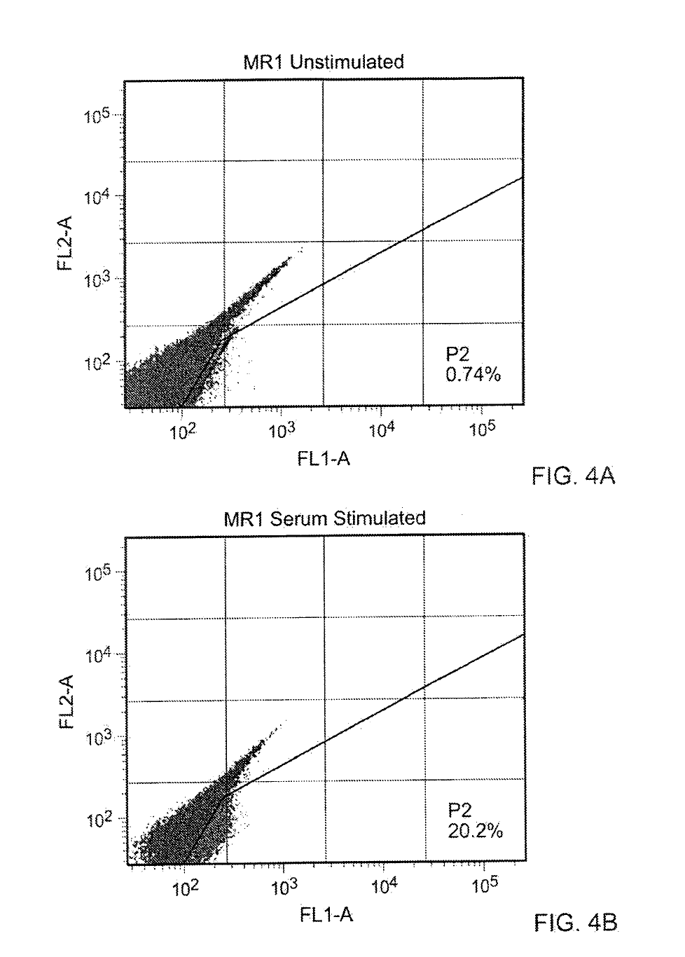

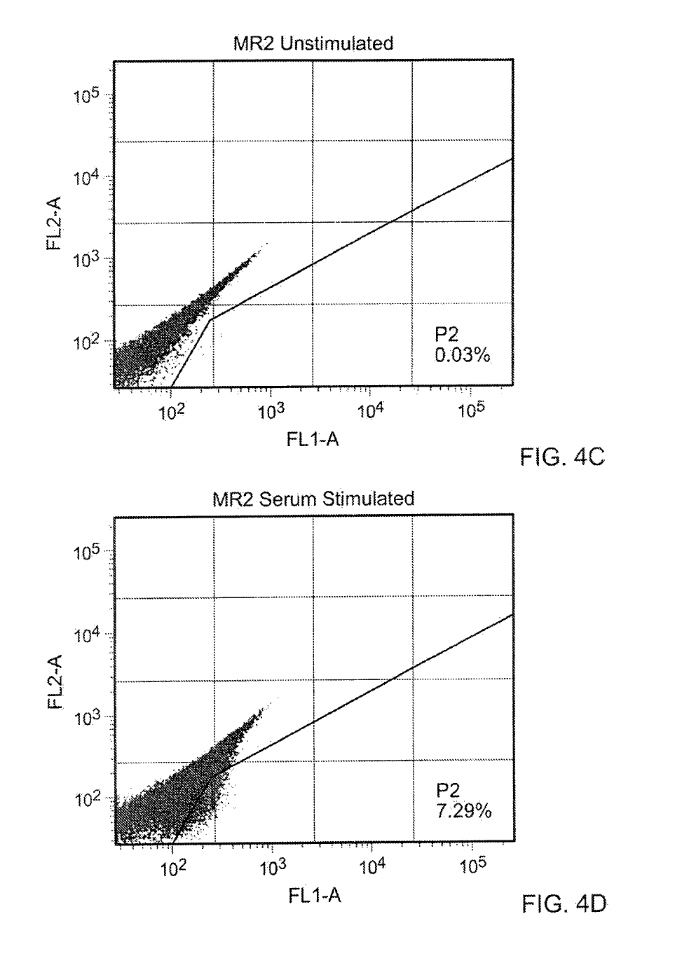

Clones 7 and 8 (designated c-Myc reporter 1 [MR1] and 2 [MR2]) were chosen for further characterization because these populations showed correct targeting and did not contain any random rAAV integration events. A cell cycle entry experiment was first performed using the same conditions used in the original selection scheme (FIG. 1). FAC sorting 6 hours following serum stimulation yielded 20.2% of the MR1 population (FIG. 4B) and 7.29% of the MR2 population (FIG. 4D) positive for EGFP-Luc. This was compared to 0.74% and 0.03% of EGFP-positive cells observed in the unstimulated MR1 and MR2 populations respectively (FIG. 4A and FIG. 4C). Thus, MR1 cells showed a 27 fold induction and MR2 cells showed a 243 fold induction of the EGFP reporter 6 hours after serum stimulation. Although there was a strong induction of EGFP, it was clear that not all cells induced the reporter by 6 hours. Therefore, the induction kinetics of the reporter gene compared to c-Myc expression during cell cycle entry of MR1 cells were further examined to determine whether the inserted EGFP reporter genes recapitulated expression characteristics of the c-Myc gene.

Induction of c-Myc transcription and protein following serum stimulation of quiescent fibroblasts peaks between 2-4 hours, and subsequently declines to low, but measurable levels by 24 hours and throughout the cell cycle (Persson et al., Mol Cell Biol. 1985 November; 5(11):2903-12, Ramsay et al., Proc Natl Acad Sci USA. 1984 December; 81(24):7742-6, Hann et al., Nature. 1985 Mar. 28-Apr. 3; 314(6009):366-9, Rabbitts et al., EMBO J. 1985 August; 4(8):2009-15, Dean et al., J Biol. Chem. 1986 Jul. 15; 261(20):9161-6). Cell cycle entry experiments were conducted using MR1 cells collected at 0, 2, 4, 8, 16, and 24 hours post stimulation. c-Myc and EGFP-Luc transcripts were induced upon serum stimulation and subsequently declined with near-identical kinetics (FIG. 5A and FIG. 5B). EGFP-Luc was also strongly induced, but its induction to measurable levels appeared to be delayed compared to c-Myc protein (FIG. 5C and FIG. 5D). It was not clear why EGFP is was not detected by 2 hour after serum stimulation, as c-Myc was, but this may have been due to the relative strength of the antibodies, or to a slower rate of translation of the EGFP-Luc fusion mRNA compared to c-Myc mRNA. EGFP-Luc protein levels also differed from endogenous c-Myc protein levels in that whereas c-Myc levels declined after 4 hours, EGFP-Luc continued to accumulate throughout the 24 hour period monitored (FIG. 5C and FIG. 5D). The progressive accumulation of EGFP-Luc was a reflection of the much longer half-life of EGFP-Luc fusion protein compared to the 20-30 minute half-life of c-Myc (Ramsay et al., Proc Natl Acad Sci USA. 1984 December; 81(24):7742-6, Hann et al., Nature. 1985 Mar. 28-Apr. 3; 314(6009):366-9., Rabbitts et al., EMBO J. 1985 August; 4(8):2009-15).

The accumulation of EGFP-Luc protein following serum stimulation was also reflected in a progressive increase over the 24 hour period in Luciferase activity (FIG. 5E) and in an robust increase in the percentage of EGFP-positive cells (FIG. 5F). Notably, the fold induction of Luciferase activity (4.5 fold) was lower that that of EGFP (10 fold) (FIG. 5E and FIG. 5F). The reason for this discrepancy is not clear, but may reflect underlying differences in the detection instruments. Importantly, the low background of EFGP-Luciferase in quiescent cells together with the progressive accumulation of EGFP-Luc signal, appeared to provide conditions that allow an amplification of events, like serum stimulation, that trigger c-Myc transcription.

A number of embodiments of the invention have been described. Nevertheless, it will be understood that various modifications may be made without departing from the spirit and scope of the invention. Other embodiments are within the scope of the following claims.

1. A method of making an indicator cell, the method comprising:
(a) contacting a vertebrate cell comprising a functional endogenous target gene under control of an endogenous inducible promoter with a parvoviral vector comprising a construct comprising a targeting DNA sequence linked to a DNA encoding a reporter gene, wherein the construct enters the cell and undergoes homologous recombination with the target gene, thereby operably linking the reporter gene and the target gene;
(b) inducing expression of the target gene thereby causing expression of the reporter gene; and
(c) selecting the cell based on expression of the reporter gene.

2. The method of claim 1, wherein selecting the cell comprises evaluating expression of the reporter gene.

3. The method of claim 2, further comprising correlating the expression of the reporter gene with the expression of the target gene.

4. The method of claim 1, wherein the reporter gene encodes a protein selected from the group consisting of: fluorescent protein, green fluorescent protein, red fluorescent protein, enhanced green fluorescent protein, luciferase, and beta-galactosidase.

5. The method of claim 1, wherein the parvoviral vector is an adeno-associated viral vector.

6. The method of claim 1, wherein the parvoviral vector is an adeno-associated viral 2 vector.

7. The method of claim 1, wherein the cell is a stem cell.

8. The method of claim 1, wherein the cell is a somatic cell.

9. The method of claim 1, wherein the cells is a mammalian cell.

10. The method of claim 1, wherein the cell is a human cell.

11. The method of claim 1, wherein the cell is selected from the group consisting of primary cell, immortalized cell, fibroblast, endothelial cell, epithelial cell, and white blood cell.

12. The method of claim 1, wherein (b) comprises contacting the cell with a compound.

13. The method of claim 12, wherein the compound is selected from the group consisting of: a small molecule, a peptide, a growth factor, a drug, and an antibody or fragment thereof.

14. The method of claim 1, wherein the target gene is selected from the group consisting of: a cell cycle gene, a DNA-damage checkpoint gene, a gene that causes cancer when overexpressed, a gene involved in cellular senescence, a gene involved in longevity and metabolism, a gene involved in apoptosis, and a gene involved in stem cell formation and function.

15. The method of claim 1, wherein selecting is performed using a method selected from the group consisting of: fluorescent-activated cell sorting, light microscopy, and drug selection.

16. An indicator cell comprising a functional endogenous target gene and an exogenous reporter gene, wherein both the target gene and the reporter gene are under control of an endogenous inducible promoter of the target gene.


SEQUENCE LISTING (12 sequences)

SEQ ID NO: 1 - DNA, 38 bp, Artificial Sequence
  Synthetically generated oligonucleotide
gggtgtcctcgcgcccgagatgcggaggaactgcgagg

SEQ ID NO: 2 - DNA, 38 bp, Artificial Sequence
  Synthetically generated oligonucleotide
agcgctaccggtcgccaccatggtgagcaagggcgagg

SEQ ID NO: 3 - DNA, 38 bp, Artificial Sequence
  Synthetically generated oligonucleotide
gtatcttaacgcggggggatcccccctcaacgttagct

SEQ ID NO: 4 - DNA, 38 bp, Artificial Sequence
  Synthetically generated oligonucleotide
cggctggatacctttcccattttcattggcagcttatt

SEQ ID NO: 5 - DNA, 29 bp, Artificial Sequence
  Primer
ggtcagatatcggaggaactgcgaggagc

SEQ ID NO: 6 - DNA, 34 bp, Artificial Sequence
  Primer
ctcggtcctgcagcatcgtcgcgggaggctgctg

SEQ ID NO: 7 - DNA, 32 bp, Artificial Sequence
  Primer
ggtcaggatcccccctcaacgttagcttcacc

SEQ ID NO: 8 - DNA, 31 bp, Artificial Sequence
  Primer
ctcggtctagagaagggatgggaggaaacgc

SEQ ID NO: 9 - DNA, 17 bp, Artificial Sequence
  Primer
gctcgcccaagtcctgc

SEQ ID NO: 10 - DNA, 20 bp, Artificial Sequence
  Primer
gctgatgtgtggagacgtgg

SEQ ID NO: 11 - DNA, 24 bp, Artificial Sequence
  Primer
tatgggctcactgagactacatca

SEQ ID NO: 12 - DNA, 21 bp, Artificial Sequence
  Primer
tcagagacttcaggcggtcaa